(19) United States
(12) Patent Application Publication
(10) Pub. No.: US 2009/0136468 A1
(43) Pub. Date: May 28, 2009

(54) PERI-OPERATIVE COMPOSITION COMPRISING LACTOBACILLUS RHAMNOSUS

(75) Inventors: Gelske Speelmans, Wageningen (NL); Adrianus Johannes Maria Vriesema, Houten (NL); Jan Knol, Wageningen (NL)

Correspondence Address: FOLEY AND LARDNER LLP;SUITE 500, 3000 K STREET NW, WASHINGTON, DC, 20007, US

(73) Assignee: N.V. NUTRICIA

(21) Appl. No.: 11/718,080

(22) PCT Filed: Oct. 28, 2005
(86) PCT No.: PCT/NL05/50026

§ 371 Date: Jun. 25, 2007

(30) Foreign Application Priority Data
Oct. 29, 2004 (EP) 04077981.1

(51) Int. Cl. A61K 35/74; A61P 41/00

(52) U.S. Cl. 424/93.45

(57) ABSTRACT

The present invention relates to the field of human health and uses of L. rhamnosus strains for the preparation of pharmaceutical and nutritional compositions.

### FIELD OF THE INVENTION

The present invention relates to enteral compositions and uses thereof comprising at least one Lactobacillus rhamnosus strain. The compositions are administered to humans before, during and/or shortly after they undergo major surgery. The compositions prevent systemic infections originating from the endogenous intestinal flora.

### BACKGROUND OF THE INVENTION

In humans undergoing major surgery, Gram-positive bacteria and/or Gram-negative bacteria, which are normally present in the intestine, can cross the intestinal wall and reach the circulation. During surgery and/or preceding surgery conditions are present, such as fasting and ischaemia which facilitate the bacterial translocation. Also the surgical procedure itself may result in a disrupted intestinal barrier. This may be the case in bowel surgery. This translocation results in short and midterm complications including sepsis, bacteraemia and endotoxaemia.

Sepsis (or septic shock or septicemia) is a disorder which occurs when a relatively large amount of micro-organisms, or fragments thereof, enter the body. It is characterised as a systemic disease associated with the presence and persistence of pathogenic micro-organisms or their toxins in the blood. If endotoxins, i.e. lipopolysaccharide (LPS) and lipoteichoic acid (LTA) and/or peptidoglycan (PG), which are bacterial fragments, are present in the blood, this condition is also referred to as endotoxaemia or endotoxic shock. When the micro-organisms which have entered the blood are viable, this condition is also referred to as bacteraemia. Lipopolysaccharide (LPS), is a component of the outer cell membrane of Gram-negative bacteria. Lipoteichoic acid (LTA) and peptidoglycan (PG) are components of the membrane of Gram-positive bacteria that can also give rise to sepsis. The intestine, especially the colon and the lower part of the small intestine, is a reservoir of LPS and Grram-negative bacteria, such as the common inhabitant Escherichia coli, but also of LTA, PG and Gram-positive bacteria. The presence of Gram-negative and/or gram-positive bacteria and/or LPS and/or LTA and/or PG in the gut is of no problem for a healthy person. However, upon increase of the intestinal permeability or decrease of the intestinal integrity during or after surgery this can become a problem. Sepsis, bacteraemia and/or endotoxaemia lead to a prolonged hospital stay and thus increased costs and increased morbidity. It can also lead to multiple organ failure or even death. It is, therefore, of great importance to find a method to treat, and especially to prevent bacterial translocation during or shortly after surgery.

Several approaches have been proposed in the prior art for the treatment of such disorders. One approach is the use of probiotic bacteria, such as lactic acid bacteria and Bifidobacteria. Especially Lactobacilli are preferred, since they are active both in the small and in the large intestine. McNaught et al., 2002 (Gut 51:827-831) and Anderson et al., 2004 (Gut 53:241-245), describe the use of strains like Lactobacillus plantarum and/or Lactobacillus acidophilus for pre-operative purposes. However, strains belonging to these species are known to form large amounts of D-lactate, which is undesirable, as will be described further below.

U.S. Pat. No. 5,591,428 discloses a strain of L. rhamnosus (DSM 6594) producing more than 5% D-lactate.

Rayes et al in Z Gastroenterol. 2002, vol 40, p 869-76 disclose that fibre and Lactobacillus plantarum, might reduce the incidence of postoperative bacterial infections. L. rhamnosus is not mentioned. Seehofer et al. in J Surg Res. 2004, vol 117(2):262-71 discloses a combination of a probiotic such as L. plantarum or L. paracasei with a fiber such as inulin or pectin which when administered orally pre- and postoperatively decreased the increased bacterial translocation in rats with simultaneous liver resection and colonic anastomosis, especially Gram-negative bacteria and enterococci. Lactobacillus rhamnosus in not mentioned.

EP 0 904 784 discloses a nutritional preparation comprising a Lactobacillus strain such as L. Rhamnosus that produces predominantly dextro-rotary lactate, Bifidobacterium and Enterococcus faecium for reducing or preventing disorders of the GI-tract. Such a Lactobacillus is used because in certain disorders levo-rotary lactate will not be digested sufficiently leading to acidosis.

U.S. Pat. No. 5,413,785 describes the use of the strain Lactobacillus rhamnosus GG to reduce the amount of endotoxins in the blood once elevated levels are found.

WO 2004/069178 describes anti-inflammatory molecules secreted by lactic acid bacteria, such as L. rhamnosus GG.

WO 93/01823 describes Lactobacillus strains able to colonise human intestinal tract mucosa, useful for treating and preventing gastrointestinal tract infections and diseases e.g. ulcerative colitis. Strains which are described as being useful for this purpose are L. plantarum 299 and L. casei ssp. rhamnosus 271.

WO 98/55131 describes pharmaceutical composition comprising Lactobacillus casei rhamnosus LB21 for the treatment of gastrointestinal disorders, especially for babies. The strain inhibits colonization by pathogenic bacteria.

The present inventors have found that L. rhamnosus strains can be used to treat or prevent sepsis, bacteraemia and/or endotoxaemia when administered to subjects which are about to undergo major surgery and/or which are undergoing and/or have been undergoing major surgery, such as open heart surgery (cardio surgery) or major abdominal surgery. Thus, known and new stains of the species of L. rhamnosus can be used in the treatment of a new group of subjects, namely subjects which have been treated by, which are and/or which will be treated by surgery, but which are otherwise healthy, especially which have a healthy intestinal microflora.

### SUMMARY OF THE INVENTION

Provided is the use of a stain of the species Lactobacillus rhamnosus which has the feature that less than 5% of the lactate produced by said strain is D-lactate, for the manufacture of an enteral composition for the treatment and/or prevention of sepsis, bacteraemia and/or endotoxaemia in humans before, during and/or after they undergo major surgery.

Preferably the % D-lactate produced by said stain is determined enzymatically, using a L-lactate acid detection kit with D- and L-lactate-dehydrogenase. It is also preferred that said Lactobacillus rhamnosus has an ampicillin and/or chloramphenicol sensitivity which is at least 2 times the sensitivity of the strain L. rhamnosus GG.

Provided is also the use of strain deposited under Accession number LMG P-22799, or any derivative thereof, for the manufacture of a medicament, especially wherein said medicament is an enteral composition for the treatment and/or prevention of sepsis, bacteraemia and/or endotoxaemia in human subjects.

Medicaments provided for therapy and/or prophylaxis are, in one embodiment, in the form of food supplements or food compositions.

### DEFINITIONS

“Sepsis, bacteraemia and/or endotoxaemia” refers to Sepsis (i.e. septic shock, septicemia or blood poisoning) which is the clinical condition in which infective agents (bacteria) or products of infection (endotoxins) enter the blood circulation and cause an overwhelming systemic inflammation. Endotoxaemia is the condition in which sepsis is caused by endotoxins. Bacteraemia is the condition whereby (living) bacteria are (transiently) present in the blood, which may cause sepsis.

“Surgical operation”, “surgical procedure” or “surgery” refers to a medical procedure involving an incision with instruments.

“Major surgery” refers to any surgical procedure that involves anaesthesia or respiratory assistance.

“Pre-operative” refers to the time period prior to surgery, such as 10 days, 7 days, 6 days, 5 days, 4 days, 3 days, 2 days, 1 day or less than 24 hours (e.g. 7 hours, 5 hours, 3 hours, 2 hours, 1 hour or less) before surgery is carried out.

“During surgery” refers to the time period when surgery is being carried out.

“Post-operative” refers to the time period posterior to surgery, such as 10 days, 7 days, 6 days, 5 days, 4 days, 3 days, 2 days, 1 day or less than 24 hours (e.g. 7 hours, 5 hours, 3 hours, 2 hours, 1 hour or less) after surgery was carried out.

“Peri-operative” refers to the time period prior, during and posterior to surgery, as defined above.

“Lactic acid bacteria” and “lactic acid producing bacteria”, is used herein interchangeably and refers to bacteria, which produce lactic acid as an end product of fermentation, such as, but not limited to, bacteria of the genus Lactobacillus, Streptococcus, Lactococcus, Oenococcus, Leuconostoc, Pediococcus, Carnobacterium, Propionibacterium, Enterococcus and Bifidobacterium.

“Probiotics” or “probiotic strain(s)” refers to strains of live micro-organisms, preferably bacteria, which have a beneficial effect on the host when ingested (e.g. enterally or by inhalation) by a subject.

The term “fibre” or “dietary fibre” is used herein to denote plant- or microbial derived food material, in particular oligo- and polysaccharides (cellulose and hemicellulose), lignin, and resistant starch, that is not digested by the human (non-bacterial) enzymes of the intestinal tract. This means that fibre is that part of the food that is not absorbed in the small intestine and thus enters in the large intestine (colon). The ‘gold standard’ method for the measurement of total dietary fibre is that of the Association of Official Analytical Chemists (2000; method 985.29).

The term “soluble” as used herein, when having reference to a polysaccharide, fibre or oligosaccharide, means that the substance is at least 50% soluble according to the method described by L. Prosky et al., J. Assoc. Off. Anal. Chem. 71, 1017-1023 (1988).

“Prebiotics” are defined as “non-digestible food ingredients that beneficially affect the host by selectively stimulating the growth and/or activity of one or a limited number of bacterial species in the colon, and thus improve the host's health (Gibson and Roberfroid 1995, J Nutr 125, 1401-1412). Commonly used prebiotics are so-called non-digestible carbohydrates (or “soluble dietary fibers”), which pass undigested through the upper part of the gastrointestinal tract into the large intestine. These include for example fructo-oligosaccharides (FOS), inulin and transgalacto-oligosaccharides (TOS).

“GOS” or “galacto-oligosaccharides”, refers to oligosaccharides composed of (preferably at least about 66%) galactose units, with a Degree of polymerization (DP) of 10 or less and an average DP of about 2, 3, 4, 5 or 6 or any value in between. A glucose unit may be present at the reducing end of the chain. “transgalacto-oligosaccharides”or “TOS” refers to a galacto-oligosaccharide in which the galactose units are linked by β-bonds.

“Degree of polymerization” or “DP” refers to the total number of saccharide units in an oligo- or polysaccharide chain. The “average DP” refers to the average DP of oligosaccharides or polysaccharide chains in a composition, without taking possible mono- or disaccharides into account (which are preferably removed if present).

A “subject” refers herein to a mammalian subject, especially a human.

“L. rhamnosus strain with the Accession number LMG P-22799” refers to the bacterial strain deposited by Nutricia N. V. under the Budapest Treaty with the Bacteria Collection Laboratorium voor Microbiologie, University Gent, K. L. Ledeganckstraat 35, B-9000 Gent, Belgium (BCCM™/LMG) on 5 Oct. 2004.

“Co-administration” of two or more substances refers to the administration of these substances to one individual, either in one composition or in separate compositions (kit of parts; as a combined composition), which are administered at the same time (simultaneously) or within a short time-span (separate or sequential use, e.g. within minutes or hours).

“Enterar” refers herein to the delivery directly into the gastrointestinal tract of a subject (e.g. orally or via a tube, catheter or stoma).

“Percentage” or “average” generally refers to percentages of averages by weight, unless otherwise specified or unless it is clear that another basis is meant. The term “comprising” is to be interpreted as specifying the presence of the stated parts, steps or components, but does not exclude the presence of one or more additional parts, steps or components. A composition comprising a L. rhamnosus strain, may thus comprise additional bacterial strains etc.

The term “a” or “an” does not limit to one, but is interpreted as at least one. The term “derivative” refers to the biological material that represents a substantially unmodified copy of the material, such as material produced by growth of micro-organisms, e.g. growth of bacteria in culture media. The term “derivative” also includes material created from the original micro-organism which retains the beneficial properties of the unmodified strain, but which is modified to have new additional properties, for example caused by heritable changes in the genetic material. These changes can either occur spontaneously or be the result of applied chemical and/or physical agents (e.g. mutagenesis agents) and/or by recombinant DNA techniques as known in the art.

Using known molecular biology methods it can be determined whether two bacterial colonies, cultures and/or preparations belong to the same strain. For example, by comparing the banding pattern observed with pulsed-field electrophoresis (PFGE) using the restriction enzymes SfiI and NotI as described by Tynkinnen et al. 1999 (Appl. Environ. Microbiol. 39:1241-1246) and by subsequently quantitatively conparing the obtained PFGE patterns by the method described in Barros et al. 2001 (1 Clin. Microbiol. 39:1241). When the similarity between the patterns is at least 95%, preferably at least 98% or more, the bacterial colonies, cultures, and/or preparations belong to the same strain. Alternatively, nucleic acid sequencing can be carried out, e.g. of conserved DNA regions.

### DETAILED DESCRIPTION OF THE INVENTION

The inventors found that, when L. rhamnosus strains were administered prior to surgery, the strains significantly reduce the translocation of bacteria of the endogenous intestinal flora across the intestinal wall under surgery conditions. A “significant reduction in translocation” refers to a statistically significant reduction in the translocation incidence and/or in the total number of bacteria translocated, compared to controls, as described in the Examples.

Whether a significant reduction of bacterial translocation occurs in vivo can be tested using animals models, for example by applying a haemorrhagic shock to fasting animals. Haemorrhagic shock is a model representing surgery conditions occurring during major cardio-surgery and major abdominal surgery.

In one embodiment L. rhamnosus strains suitable for manufacturing enteral compositions for the treatment and/or prevention of sepsis, bacteraemia and/or endotoxaemia in humans are provided, whereby such compositions are administered before and/or during and/or after the human subject undergoes major surgery, i.e. pre-operatively, during surgery, post-operatively and/or peri-operatively. The compositions are preferably not given before, during and/or after types of major surgery which involve suppression of the immune system of the subject (for example as generally applied during liver transplants and the like). Hunan subjects having a suppressed immune system are preferably not administered compositions comprising probiotics. Any L. rhamnosus strain is in principle suitable. However, in a preferred embodiment the strain used produces predominantly, or even exclusively levo-rotary lactate (also referred to as L-lactate or L-(+)-lactate). In a preferred embodiment of the lactate produced by the strain, less than 5%, preferably less than 3%, more preferably less than 2% is dextro-rotary-lactate (also referred to as D (−)-lactate or D-lactate). The percentage of D-lactate produced can be determined as known in the art, for example by analysing supernatant of an culture of the Lactobacillus strain grown to stationary growth phase in a medium to which a carbohydrate source lactose (or glucose) was added. Of course, the micro-organism can produce other metabolites besides the lactate. The use of these strains is preferred because in certain disorders of the gastrointestinal-tract, such as short bowel syndrome, carbohydrate mal-absorption or in cases of carbohydrate overload, D (−)-lactate will not be digested sufficiently fast enough, which can cause adverse reactions such as acidosis.

In another embodiment L. rhamnosus strains, which are especially suitable for the preparation of the above compositions, are selected based on one or more selection criteria. These criteria include the analysis of one or more of the following characteristics and/or use of one or more of the following assays:

a) the “Dynamic gastro-intesfinal model assay”, as described by Marteau et al. 1997 (J Dairy Sci. 80:1031-1037) and in the Examples. In this assay, strains which have a survival which is above 25%, preferably above 30%, more preferably above 35% or 40% are particularly suitable. The percentage survival is above that of the control strain L. rhamnosus GG preferably at least 5%, 10%, 15%, 20%, 30%, 40%, 50% or more above the survival of L. rhamnosus GG (ATCC 53103).

b) antibiotic sensitivity/resistance may be used to select strains with a higher or with an increased sensitivity to one or more antibiotics which are generally used parenterally to prevent and/or treat systemic infections caused by the human enteric microflora (since this enables an interference by antibiotic treatment when necessary). Preferably, the sensitivity to ampicillin and/or chloramphenicol is tested, but other antibiotics may be tested as well (see Examples).

In order to select such strains, they are grown on media comprising the antibiotic and the minimal inhibitory concentration (MIC) is determined. The MIC refers to the lowest concentration of the antibiotic tested, which results in no bacterial growth after 24 hours at 37° C. Preferably, the minimal inhibitory concentration for ampicillin and/or chloramphenicol is less than 10 μg/ml, preferably less than 6 μg/ml, less than 5 μg/ml or even less. In a preferred embodiment the strain shows a sensitivity to ampicillin and/or chloramphenicol which is at least 1.5 times, preferably 2 times higher (or more) than that of the control strain Lactobacillus GG (ATCC 53103).

c) it is preferred that strain selected do not comprise a capsule (or slime layer) outside the cell wall. Capsule formation can be determined as described by Cappucino and Sherman “Microbiology—A laboratory Manual”, 4^{th} edition, p 75.

d) the percentage inhibition of adhesion of gram-negative pathogens (especially Klebsiella pneumoniae strain LMG 21902) to intestinal cells, such as Caco-2 cells, is preferably at least 10%, 15%, 20% or more higher than the inhibition of adhesion caused by strain L. rhamnosus GG (ATCC 53103). The inhibition can be tested as described in the Examples and by Coconnier et al. 1993 (FEMS Microbiol Lett. 110(3):299-305).

and/or

e) shelf-life properties. Shelf life properties can be determined as described in the Examples (see Example 7 and FIG. 1), e.g. by % survival after storage at room temperature over several weeks or months. As an alternative, the shelf life experiment can be carried at 37° C. instead of room temperature. This enables an enhanced determination of shelf life properties and comparison of shelf life properties between strains within 4 weeks instead of 6 to 12 months. Survival of freeze-dried strains (e.g. L. rhamnosus LMG P-22799 or others as selected using this characteristic) in a dried, powdered product, is preferably at least 20%, 30%, 40%, 50% or more better than survival of the control strain L. rhamnosus GG (ATCC 53103) after 26 weeks of storage at room temperature and/or after 2 weeks of storage at 37° C.

These assays enable identification of strains with superior probiotic properties, such as GI-tract survival, antibiotic resistance and/or process-technological properties, and with suitable characteristics for the manufacture of pre-, post and/or peri-operative compositions.

Compositions for the treatment or prevention of sepsis, bacteraemia and/or endotoxaemia in humans comprise at least one L. rhamnosus strain, preferably a strain with one or more of the above properties. The compositions may obviously comprise other components, such as carriers (e.g. carbohydrates such as maltodextrin), etc., as described elsewhere herein. In one embodiment the L. rhamnosus strain is strain LMG P-22799.

In yet another embodiment, a composition comprising at least one L. rhamnosus strain, and further comprising one or more of the following is provided: a Bifidobacterium strain (preferably a strain belonging to the species B. longum), a Saccharomyces species preferably a strain belonging to the species Saccharomyces cerevisiae), a further Lactobacillus strain (preferably of the species L. plantarum) and/or a soluble fiber (preferably TOS). When administered pre-, during, post- and/or peri-operatively such compositions significantly reduce bacterial translocation. The L. rhamnosus strain may be any L. rhamnosus strain or a strain selected as described above. In one embodiment the L. rhamnosus strain is strain LMG P-22799. It is understood that the components of the compositions according to the invention may be supplied as separate units, which are co-administered.

The compositions according to the invention may be pharmaceutical compositions or food or food supplement compositions, suitable for enteral, preferably oral, administration. The compositions can be in a form for separate administration, such as a capsule, a tablet, a powder, a gel, or a similar form. The dosage form comprises preferably a unit dose of the L. rhamnosus strain. Suitable dosages are 1×10⁶ to 1×10¹², preferably 1×10⁸ to 5×10¹⁰ colony forming units per dose, or the equivalent dosage of non-viable or dead cells. If the strain is non-viable the dosage is determined while the strain is still alive/viable. As already mentioned, in one embodiment the compositions may further comprise one or more additional micro-organisms in suitable amounts, such as at least a Bifidobacterium (selected from B. breve, B. longum, B. lactis, B. animalis, B. adolescentis, B. infantis and B. bifidum), a L. plantarum strain and/or a yeast strain (e.g. of the genus Saccharomyces).

The food supplement composition can be in the form of a powder or a similar form, which is added to, or mixed with, a suitable food (composition) or a suitable liquid or solid carrier, for the preparation of a food which is ready for consumption.

A preferred embodiment is a freeze-dried powder of the L. rhamnosus strain (and optionally one or more further micro-organisms as described), which can be in the form of a sachet, or which can be incorporated in a capsule or a tablet or another dry administration form. These freeze-dried preparations can be obtained using known techniques and can contain suitable adjuvants known per se, for instance cryoprotectants such as maltose.

For instance, the invention in the form of a food supplement can be in the form of a freeze-dried powder, which is reconstituted using a suitable liquid, such as water, oral rehydration solution, milk, fruit juice, or similar drinkable liquids. It can also be in the form of a powder which is mixed with solid foods, or foods with a high water-content, such as fermented milk products, for example yoghurt.

The nutritional preparations of the invention can also be in the form of a food which is ready for consumption. Such a food can, for instance, be prepared by adding a supplement of the invention as described above to a food or food base known per se, or by adding the micro-organism(s) (separately or as a mixture) in the amounts required for administration to a food or food base known per se; or by cultivating the required bacteria in a food medium until a food containing the amount of bacteria required for administration is obtained. This medium is preferably such that it already forms part of the food, or will form part of the food after fermentation. In this respect, the nutritional composition can be either fermented or non-fermented.

The nutritional composition according to the invention may be a food for oral consumption, for instance a total food; or a food or food supplement for administration by tube or catheter into the stomach or the gut. The tube can be nasooesophageal, nasogastric or nasojejunal, but also gastrostomy or jejustomy tubes are envisaged. The oral feeding can be a complete feeding or a food supplement, in liquid form or as a capsule or powder. Preferred liquid forms are dispersions.

The nutritional preparation of the invention can further comprise all desired components, and/or additives which are suited for use in food or food supplements, including flavours, colourings, preservatives, sugar etc. When live or viable micro-organisms are used it is preferred that the further components do not affect the viability of the micro-organisms present therein. Some preferred specific additives are discussed herein below.

In compositions comprising fibres, the fibres used may be in an amount of at least 0.5 g per 100 g of the total composition. When formulated as a food for tube feeding, the composition of the invention preferably contains 0.2 to 25 g fibres per 10⁸ to 10¹¹ cfi L. rhamnosus present in the composition; a dry supplement according to the invention preferably contains 0.25 to 50 g fibres per 5×10⁸ to 10¹⁰ L. rhamnosus present in the composition. In another embodiment the fibre composition comprises at least 50 wt. %, preferably at least 65 wt. % soluble fibres based on total fibre content. Preferably the composition comprises at least 20 wt. % prebiotics based on total fibre content.

Suitable prebiotics for use herein are selected from galacto-oligosaccharides, trans-galacto-oligosaccharide (TOS), partially hydrolysed galactomannan (e.g. hydrolysed guar gum), inulin, hydrolysed inulin, fructooligosaccharides, indigestible dextrin, pectin oligosaccharides, and mixtures thereof. A preferred prebiotic is trans-galacto-oligosaccharide, since L. rhamnosus grows best on this substrate. As a result, the likelihood that the L. rhamnosus cells reach the intestines alive increases. Further, the combination of prebiotic with the probiotic enable increase of the beneficial action of the growth of beneficial, non-translocating intestinal bacteria such as Bifidobacteria. Accordingly, prebiotics can advantageously be used in composition according to the invention.

The composition, in particular when in the form of a total food, may also comprise peptides and/or proteins, in particular proteins that are rich in glutamate and glutamine, lipids, digestible carbohydrates, vitamins, minerals and trace elements. The use of glutamine/glutamate precursors, in amounts corresponding to 0, 6-3 g glutamine/100 g product, as well as of small polypeptides that contain a high amount of glutamines, is preferred. Alternatively, proteins that are rich in glutamine, such as milk proteins, wheat proteins or hydrolysates thereof, may be present.

When present, the digestible carbohydrate may be any suitable digestible carbohydrate or digestible carbohydrate mixture. For example, the digestible carbohydrate may be glucose, sucrose, maltodextrin, modified starch, amylose starch, tapioca starch, corn starch, corn syrup, or fructose, or mixtures thereof. Maltodextrin is preferred if low osmolarity is required.

The compositions according to the invention are preferably low in lactose i.e. have a lactose concentration below 3 g per dose. In a preferred embodiment the composition contains 5 to 230 g digestible carbohydrates. Digestible carbohydrates are an excellent source of energy. During or after surgery, the body's energy demands are high. Carbohydrates supply this energy to provide strength for the healing. Therefore, digestible carbohydrates and L. rhamnosus may act synergistically.

The compositions preferably do not have a high osmolarity preferably less than 400 mosm/l, more preferably less than 300 mosm/l) since a high osmolarity results in less gastrointestinal problems such as diarrhoea.

All micro-organisms used in the invention are preferably resistant to degrading conditions in the gastrointestinal tract, such as amylase, stomach acids, bile (salts), lipases and/or pancreatic fluid, so that they can pass the stomach and have a beneficial influence on the gastrointestinal tract. Therefore, they can be used as such without applying any additional measures in order to protect the micro-organisms during their passing the stomach. In from the stomach environment by encapsulation. Encapsulation of the micro-organisms can provide an improved shelf-life of two years or more, in particular for food products which are ready for consumption. They also improve the safety of supplements for adding to food products prior to consumption. Also, such encapsulation can even further improve the resistance against stomach fluids and/or pancreatic fluid.

The probiotic micro-organisms, especially the L. rhamnosus strain used, adhere to the walls of the gastrointestinal tract, in particular to the mucous membranes and/or receptors thereon, and thereby compete with the endogenous micro-organisms, such as the endogenous Gram-negative bacteria under the dynamic conditions present in the GI-tract. This prevents that the bacteria translocate the intestinal barrier, and also speeds up clearance of the pathogens from the gastrointestinal tract. This prevention of adherence (of both living and/or dead pathogens, as well as fragments thereof) can be increased considerably by including anti-adhesive agents in the compositions. By including specific fibres, the beneficial micro-organisms will have sufficient substrate available for growth and produce factors that can heal the damaged gut tissue.

The compositions according to the invention have a positive influence on the gastrointestinal tract. For this purpose, they may further comprise health improving compounds known per se, such as medicaments or other bioactive compounds, etc. In particular, the preparations may comprise compounds which have a beneficial influence on the gastro-intestinal tract, such as glutamine/glutamate or precursors thereof.

The enteral composition of the invention is preferably used in the prevention and/or treatment of sepsis, bacteraemia and/or endotoxaemia (endotoxic shock) and to delay acute phase response. In particular the composition is used for (prophylactic) treatment of patients receiving major surgery, critically ill patients, patients with Inflammatory Bowel disease (IBD), pouchitis, patients with HELLP syndromes, patients with food allergy, patients with an enhanced risk for bacterial translocation and sepsis in general, in particular those suffering from major trauma, burns, pneumonia, especially caused or complicated by bacteria, decubitus, during radio/chemotherapy or patients having a compromised immune system such as premature infants or people suffering from elderly diseases, or people with an obstructed bile duct. In a particular preferred embodiment the composition is used for treatment and/or prevention of sepsis, endotoxaemia and/or bacteraemia in patients receiving major surgery before, during and/or after the surgical procedure.

In case of treatment and/or prevention of sepsis, endotoxaemia, and/or bacteraemia in surgical patients, the composition is preferably administered during a period of at least 12 hours, preferably at least 1 day, more preferably at least 4 days, even more preferably at least 7 days prior to the surgical procedure. Preferably the composition is used at most 3 weeks prior to the surgical procedure. The composition is preferably administered during a period of at least 12 hours, preferably one day, more preferably at least 4 days, even more preferably at least 7 days after the surgical procedure. Preferably the composition is administered during a period of at most 3 weeks after the surgical procedure.

The composition is preferably administered in one, two or three doses per day. Alternatively, the composition may be administered continuously via tube feeding, in which case this form of administration counts as one daily dose. The daily dose is the single dose multiplied by the number of doses which are taken within 24 hours.

Without limiting the invention, it is believed that the risk of endotoxaemia is related to the fact that patients need to fast pre-operatively and that after surgery they take-in only little food. As a result, malnutrition and increased intestinal permeability occurs, which makes them more vulnerable to bacterial translocation. Generally these patients can take the composition of the invention until about 3 hours before surgery and shortly after surgery, but also the option of taking the composition during a shorter period before surgery is envisaged by the invention. Preferably, the composition is taken each day in two daily doses, starting one week in advance. Also ischaemic conditions of the intestine which are common during major surgery and the surgical introduction of intestinal wounds during some forms of bowel surgery result in increased intestinal permeability.

The species identity of micro-organisms can be determined biochemically or by sequencing (e.g. conserved regions) or by known methods such as pulse field gel electrophoresis. In general, strains of bacteria belong to the same species if they show at least 97% nucleic acid sequence identity in the 16 S rRNA region (e.g. when optimally aligned by for example the programs GAP or BESTFIT using default parameters).

### FIGURE LEGENDS

FIG. 1: Survival of Lactobacillus rhamnosus LMG P-22799 (□) and Lactobacillus rhamnosus GG (ATTC 53103) (▪) in time in a powdered infant milk formula (IMF) stored at room temperature. The different lines represent different batches of IMF.

The following non-limiting Examples describe the uses and methods according to the invention. Unless stated otherwise, the practice of the invention will employ standard conventional methods of molecular biology, pharmacology, immunology, virology, microbiology or biochemistry. Such techniques are described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and Remington's Pharmaceutical Sciences, Mack Publishing Company, Philadelphia, Pa., 17th ed. (1985), Microbiology: A Laboratory Manual (6th Edition) by James Cappuccino, Laboratory Methods in Food Microbiology (3^{rd} edition) by W. Harrigan (Author) Academic Press, all incorporated herein by reference.

### EXAMPLES

### Example 1

### Method of Selection of L. rhamnosus Strain LMG P-22799

### 1.1 Isolation

A large number of Lactobacilli, including strains isolated from faces of healthy adult human volunteers or from traditionally fermented foods, were subjected to a selection procedure.

Fresh human faces or traditionally fermented food was analysed in an anaerobic chamber. The faces was diluted tenfold in 90 ml of storage medium (20 g/l buffered peptone water, 1.0 ml/l Tween 80, 0.5 g/l L-cysteine-HCl and 1 Resazurin tablet per liter, pH 6.3 (adjusted with 2M HCl)) and then homogenised by using an Ultra-Turrax. Serial dilutions were made in reduced physiologic pepton water and the 102-107 dilutions are plated on LAMVAB (Hartemink et al. 1997, LAMVAB “A new selective medium for the isolation of lactobacilli from faces, 3. Microbiological methods 29, 77-84). The low pH (5.0) in this medium inhibits the growth of Gram-negative bacteria, whereas the Gram-positive L(+)-lactate producing Lactobacilli are resistant to vancomycin, so LAMVAB is selective for these strains. This medium consist of 104.4 g/l De Man, Rogosa and Sharpe (MRS, Oxoid), 0.5 g/l L-cysteine-HCl, 0.05 g/l bromocresol green, 40 g/l agar, and 20 mg/l vancomycin. MRS, L-cysteine-HCl and bromocresol green were autoclaved separately from the agar for 15 minutes at 121° C. and cooled down to 50° C. Vancomycin was sterilised by filtration using a 0.2 μm filter. The three liquids were mixed together and plates are poured. The plates are incubated at 37° C. in anaerobic jars for three days. Gram-positive, catalase-negative rod-shaped bacteria isolates are streaked for purity on MRS agar and incubated at 37° C.

### 1.2 Species Identification

The API 50CHL (BioMerieux SA, France) is used for tentative identification of the strains by their fermentation profiles. Cells are grown overnight on MRS agar plates. Cells are removed from the agar plate with a sterile swab and resuspended in the suspension medium provided by the kit. API-strips are inoculated and analysed after 24 and 48 hours. About 250 strains showed characteristics of a Lactobacillus rhamnosus, L. paracasei, or L. casei species. These strains were selected for the furhter selection procedure.

### Selection Procedure:

L. rhamnosus, L. paracasei and L. casei strains were selected based on their survival under GI-tract conditions production of L(+)- and D(−)-lactate, growth on commercially available prebiotic substrates, adhesion to Caco-2 cells and prevention of adhesion of non-invasive Gram-negative bacteria, antibiotic resistance, and the process technological property shelf life and their EPS and capsule producing properties. The strain showing the most superior characteristics was confirmed to be a strain of L. rhamnosus, with 16 sRNA sequencing and deposited at BCCM™, Gent, Belgium under the accession number LMG P-22799.

1.3 16S rRNA

Sequencing of the 16S rRNA gene gives a reliable identification of the strains. The extraction of the DNA of the strains is done according to the method described by Walter et al., 2000, “Detection and identification of gastrointestinal Lactobacillus species by using denaturing gradient gel electrophoresis and species-specific PCR primers”, Applied and Environmental Microbiology, 66 (1), 297-303. The amplification and sequencing of the 16S rRNA region is accomplished with primers mentioned in Table 1. The amplification program is 94° C. for 5 min; 30 cycles of 94° C. for 30 s, 54° C. for 30 s, 72° C. for 1 min 30 s; and finally 72° C. for 4 min.

TABLE 1
Sequence primers

| | |
|---|---|
| Sequence Primer | Sequence (5′ → 3′) |

| | |
|---|---|
| 8f | CAC GGA TCC AGA GTT TGA T(C/T)(A/C) TGG CTC AG |
| 338r | GCT GCC TCC CGT AGG AG |
| 338f | CTC CTA CGG GAG GCA GC |
| 515f | TGC CAG CAG CCG CGG TAA TAC GAT |
| 515r | ATC GTA TTA CCG CGG CTG CTG GCA |
| 968f | AAC GCG AAG AAC CTT AC |
| 968r | GTA AGG TTC TTC GCG TT |
| 1401r | CGG TGT GTA CAA GAC CC |
| 1510r | GTG AAG CTT ACG G(C/T)T ACC TTG TTA CGA CTT |

Sequencing is carried out by the dideoxy method of Sanger et al., 1977, “DNA sequencing with chain-terminating inhibitors”, Proc. Natl. Acad. Sci. USA 74, 5463-5467, by using the ABI PRISM BigDye Terminator Cycle Sequencing Ready Reaction kit (Applied Biosystems Inc., Nieuwerkerk aan de IJssel, Netherlands) in combination with Applied Biosystems model 310 automated sequencing system. Analysis of nucleotide sequence data is carried out by using the Chromas and DNAsis program. The strain is identified with a BLAST search (NCBI), searching in the GenBank, EMBL, DDBJ and PDB databases.

### 1.4 L- and D-Lactic Acid Assay

Lactobacillus strains were cultured overnight in MRS-broth at 37° C. Cells were harvested by centrifugation at 4000 rpm in a RT7 Sorvall centrifuge. Supernatants were stored at −20° C., until analysis.

Lactate was determined enzymatically, using a L-lactate acid detection kit with D- and L-lactate-dehydrogenase (Boehringer Manheirn, Mannheim, Germany) (according to the instructions given by the supplier of the kit).

L. rhamnosus LMG P-22799 Produced 98% L-Lactate and 2% D-Lactate.

### Example 2

### Survival In Vivo

The survival in the stomach and small intestine of L. rhamnosus LMG P-22799 as well as other known probiotic strains was evaluated. The survival of the strain in the stomach and small intestine is important when the strain is used as a probiotic in humans. Besides L. rhamnosus LMG P-22799 also Lactobacillus GG (ATCC 53103) was tested in a dynamic model of the stomach and small intestine as described in Marteau et al, 1997, J Dairy Sci 80:1031-1037. This model allows simulation of successive in vivo conditions in the stomach and small intestine, such as the kinetics of the pH, bile salt concentrations, and transit of chyme, and peristalsis and is an excellent model predicting the survival of the bacteria in vivo (in humans).

The Lactobacillus rhamnosus strains were grown in MRS for 24 hours and subsequently re-inoculated for 18 hours in MRS. Cells were harvested by centrifugation and washed with peptone physiological salt (PFZ, 8.5 g/l NaCl and 1.0 g/l neutralised bacteriological peptone). Bacterial cells were resuspended in sterile skimmed milk (at a concentration of about 10⁷ colony forming units (cfu)/ml) and about 10⁹ cfu/ml were added to the model. The two strains were tested simultaneously. During 6 hours every hour a sample was taken out of the model and plated out on MRS to calculate the amount of viable cells.

The model was set to resemble the in vivo conditions of the human digestion of milk. The pH curve in the stomach was computer controlled: pH 6.5 at initiation, pH 4.2 at 20 min, pH 2.8 at 40 min, pH 2.1 at 60 min, and pH 1.8 at >90 min. In the small bowel compartments the pH was kept at 6.5 in the duodenum, 6.8 in the jejunum, and 7.2 in the ileum.

Gastric emptying was set at a half time of 30 min and the β coefficient of the power exponential equation was 1. For ileal emptying the half time was 160 min, and the β coefficient was 1.6.

At t=0 10 ml of stomach solution, containing 0.28 mg/ml pepsin and 3.75 mg/ml lipase, was added to 150 ml of milk/Lactobacillus suspension, subsequently the stomach solution was added to the stomach compartment with a constant flow of 0.25 ml/min, As a pepsin source, porcine stomach pepsin (Sigma Chemicol Co P6887), was used. As a lipase source (lipase F-AP15 Amano Pharmaceutical Co, Ltd Nagoya), was used. In addition salt solution and acid were added to obtain a total stomach secretion of 0.50 ml/min.

As a bile source a bovine bile extract, (Sigma Chemicol Co B3883), was used. At t=0 the duodenum contained a solution of 5.6 mg/ml, subsequently a solution of 16.7 mg/ml bile was added with a constant flow of 0.5 ml/min to the duodenum compartment. As a pancreatin source, a porcine pancreatin extract, (Sigma Chemicol Co p1750), was used. At t=0 the duodenum contained a 1.2% pancreatin solution, subsequently a 7% pancreatin solution was added with a constant flow of 0.25 ml/min to the duodenum compartment. In addition salt solution and base were added to obtain a total duodenum secretion of 1.0 ml/min. Salt, acid and base concentrations were the same as described by Marteau et al. (1997, supra).

As can be seen in Table 2, strain LMG P-229799 showed a better survival under GI tract conditions than L. rhamnosus GG.

TABLE 2
The survival of some Lactobacillus strains
in the dynamic stomach-intestine model.

| | Survival dynamic stomach small |
|---|---|
| | intestine model (% of added total |
| Strain | number of bacteria) |
| L. rhamnosus LMG-22799 | 40 |
| L. rhamnosus GG | 22 |
| ATCC 53103 | |

### Example 3

### Inhibition of Adhesion of Enteral Gram-Negative Bacteria by L. rhamnosus LMG P-22799

Interaction between pathogenic bacteria and the host cells initiates infectious diseases. Attachment of these pathogens to intestinal cells is the first step of an infection. The pathogenic bacteria may colonise, cause cell damage and when invasive cross the epithelial membrane. Probiotic strains and pathogens compete with each other on the binding sites of epithelial cells. Probiotic strains can prevent the pathogens in binding to the intestinal cells.

Using cultured Caco-2 cells as a human intestinal cell model, the adhesion of pathogens and probiotic strains to cells (Coconnier, et al. 1993, supra) was tested. Pathogens tested were Klebsiella pneumoniae LMG 21902 and Pseudomonas aeruginosa LMG 21901, both deposited at the BCCM™/LMG. The probiotics used were L. rhamnosus LMG P-22799 and the commercial used strain Lactobacillus rhamnosus GG (ATTC 53103).

Overnight cultures of pathogens and probiotics were harvested by centrifugation (10 minutes, 4000 rpm, Sorval RTI 7) and resuspended in PBS. The amount of cells was counted under a microscope with use of Bürker Türk counting chamber. Bacteria were centrifuged again and the pellet was resuspended in Caco-2 1% FCS-medium Pen/Strep free. The Caco-2 cells were 2 weeks post-confluence and grown in a 24 wells-plate (2.5 10⁵ Caco-2 cells per well). Per well 5×10⁷ CFU of the pathogens and 2.5×10⁸ CFU of the probiotic bacteria were added and incubated for 1 hour at 37° C. in an incubator with 5% CO₂. After incubation the media was removed from the Caco-2 cells and the cells were washed 3 times with PBS (37° C.). Cells were lysed with sterile MiliQ water, serial dilutions of the lysed cells were made and plated on Nutrient agar (for pathogens) and on MRS agar (for probiotic strains).

L. rhamnosus LMG P-22799 was found to reduce the adherence of the pathogens to intestinal cells better than L. rhamnosus GG (Table 3).

TABLE 3
Inhibition of adhesion of pathogens
to Caco-2 cells by probiotics (%)

| | K. pneumonia | P. aeruginosa |
|---|---|---|
| Strain | LMG 21902* | LMG 21901* |
| L. rhamnosus | 54 | 38 |
| LMG P-22799 | | |
| L. rhamnosus GG | 44 | 38 |
| (ATTC 53103) | | |

*The prevention (%) of the adhesion of the Caco-2 cells by probiotic bacteria: Difference in adherence of the pathogens with and without probiotic strains.

### Example 4

### Capsule Analysis

Capsule determination was performed as described in Cappucino James G., Sherman Natalie; MICROBIOLOGYA laboratory manual; 4^{th} edition; P 75. The lactobacilli were pre-cultured anaerobically in 10 ml MRS broth in 50 ml Falcon tubes at 37° C. in jars. A capsule is a gelatinous outer layer that is secreted by the cell and that surrounds and adheres to the cell wall. Cells that have a heavy capsule are generally virulent and capable of producing disease, since the structure protects bacteria against the normal phagocytic activities of host cells. It is, therefore, unwanted that a prebiotic strain used for peri-operative purposes contains a capsule. As a positive control Klebsiella pneumoniae LMG 21901 was used Strain LMG P-22799 did not contain a capsule.

### Example 5

### In Vivo Assays in Rat Models

### 5.1 Animals

The protocol of this study was approved by the Animal Care Corrmittee of the University of Maastricht, the Netherlands. Healthy male Sprague-Dawley rats, weighing 319-403 grams (average, 364 grams) purchased from Charles River (Maastricht, the Netherlands) were housed under controlled conditions of temperature (20-22° C.) and humidity (50%). Before the beginning of the experiments, rats were fed water and chow ad libitum.

### 5.2 Experimental Design

Animals were divided into 3 groups (n=6 per group). One group was normally fed prior to the surgical intervention and received sham shock (SS-C). The haemorrhagic shock control group (HS-C) was normally fed prior to the surgical procedure. A third group received during 7 days preceding the haemorrhagic shock per oral gavage a single daily dose of maltodextrin, which is the carrier of the probiotic composition, (HS-M). The probiotic test group received during 7 days preceding haemorrhagic shock a single daily dose of 5*10e9 cfu of L. rhamnosus LMGP-22799 in maltodextrin (HS-Lr). All rats were starved 18 hours before the procedure. Blood and tissue samples were taken at 24 hours after onset of the (sham) shock.

### 5.3 Haemorrhagic Shock Procedure

Rats were anesthetised with intraperitonally injected sodium pentobarbital (40 mg/kg). The skin over the left femoral area was shaved and disinfected with povidone iodine solution. The animals were placed in the supine position and allowed to breathe spontaneously. During surgery and throughout the experiment, body temperature was maintained at 37° C. with an infrared heating lamp controlled by a thermo analyser system (Hugo Sachs Elektronik, March-Hugstetten, Germany) connected to a rectal probe. The femoral artery was dissected using aseptic technique and cannulated with polyethylene tubing (PE-10) containing heparinised saline (10 IU/ml). Arterial blood pressure was continuously measured (Uniflow™ external pressure transducer; Baxter™, Utrecht, the Netherlands) and recorded as Mean Arterial Pressure (MAP). Heart rate (HR) was continuously assessed from the instantaneous pressure signal. To keep the arterial catheter patent, it was constantly perfiised with physiological saline (3 ml/h) via the Uniflow™ system; no heparin was used After an acclimatisation period of 30 minutes, rats were subjected to haemorrhage by withdrawing blood in quantities of 2.1 ml/100 gram of bodyweight (representing approximately 30-40% of the circulating volume) at a rate of 1 ml/minute. At 50 minutes after the induction of shock, the catheter was removed and the femoral artery ligated.

Six hours after hemorrhage, the animals were allowed access to standard chow ad libitum. Rats in the sham-shock group were anesthetised and the left femoral artery was cannulated. Sham shock rats were monitored similar as the haemorrhagic shock group, however no blood was withdrawn.

Twenty-four hours after induction of (sham) shock, the rats were anesthetised with sodium pentobarbital (60 mg/kg). The skin over de abdomen was shaved and disinfected with povidone iodine. The abdomen was opened via a midlne incision, blood samples were taken and mesenteric lymph nodes, the midsection of the spleen and segment IV of the liver were aseptically removed for bacteriological examination.

### 5.4 Bacterial Translocation

Mesenteric lymph nodes (MLN), the mid-section of the spleen and a segment of the liver were collected aseptically in 2 ml pre-weighed thioglycolate broth tubes (Becton Dickinson (BBL) Microbiology Europe, Maylan, France). After weighing, the tissue specimens were homogenised with sterile grinding rods (Potter S, B. Braun Melsungen, Melsungen, Germany). Subsequently, 500 μl-volumes were transferred onto the following agar plates: Columbia III blood agar base supplemented with 5% vol/vol sheep blood (BBL) (duplicate plates), Chocolate PolyviteX agar (BioMérieux, Marcy L'Etoile, France), and Schaedler Kanamycin-Vancomycin agar supplemented with 5% sheep blood (BBL). Aliquots were spread over the entire surface of the agar. All agar plates were incubated for 48 h, in a 5% CO2-enriched atmosphere or under anaerobic conditions (Shaedler agar plates). After incubation, the colonies were counted on the non-selective Columbia sheep blood agar plates. For determination of the number of colony forming units (CFU) per gram tissue, the number of colonies was counted on all aerobic plates and next adjusted to the weight of the grounded tissue. Colony types were identified to the species level using conventional techniques.

### 5.5 Statistical Analysis

Bacterial translocation data are represented as median and range; other data are represented as mean±SEM. A non-parametric Mann-Whitney U test was used for between-group comparisons.

### 5.6 Results

Table 4 shows the results regarding the bacterial numbers translocated, wherein the mean value and range (between brackets) are presented.

TABLE 4
Translocation incidence (TI) and bacterial numbers translocated as mean
value (range) obtained from a non-parametric Mann-Whitney U test

| | TI | MLN | | Spleen | | Liver | | Total |
|---|---|---|---|---|---|---|---|---|
| SS-C | 2/6 | 0 | (0-33) | 0 | (0-17) | 0 | (0-95) | 0 |
| (n = 6) | | | | | | | | |
| HS-C | 6/6 | 300 | (114-483) | 12 | (0-998) | 34 | (0-249) | 369 |
| (n = 6) | | | | | | | | |
| HS-M | 6/6 | 109 | (74-262) | 33 | (14-832) | 27 | (7-161) | 292 |
| (n = 6) | | | | | | | | |
| HS-Lr | 3/6 | 2 | (0-37)*# | 0 | (0-11)*# | 0 | (0-6)*# | 2#* |
| (n = 6) | | | | | | | | |

*p < 0.05 compared to HS-C
#P < 0.05 compared to HS-M

Starved rats receiving a haemorrhagic shock (HS-C) or a haemorrhagic shock after administering the carrier maltodextrin (HS-M) showed significantly higher bacterial translocation numbers compared to rats receiving a composition containing L. rhamnosus LMG P-22799 (HS-Lr). Also translocation incidence was affected A composition with L. rhamnosus LMG P-22799 had a positive effect on the incidence of bacterial translocation.

The results obtained demonstrate that L. rhamnosus LMG P-22799 protects against bacterial translocation occurring as a result from surgery and hemorrhage both on the level of incidence as on the level of bacterial numbers translocated. Reduction of incidence of bacterial translocation and reduction in the number of bacteria translocated reduces the change on bacteraemia, and reduces the influx of LPS and LTA and thereby thus the chance for development of endotoxaemia and sepsis.

### Example 6

### Compositions

### 6.1 Sachet Comprising LMG P-22799

A composition is described for use in surgical patients prior to, during and/or after surgery containing a powder with the following composition:

| L. rhamnosus LMG P-22799 | 10¹⁰ | cfu |
|---|---|---|
| L. plantarum | 10¹⁰ | cfu |
| B. longum | 10¹⁰ | cfu |
| S. cerevisiae | 0.5 | g |
| Fibers | 22.75 | g |
| Soy polysaccharides | 7.2 | g |
| Cellulose | 2.7 | g |
| Gum Arabic | 9.45 | g |
| Fructo-oligosaccharides (Raftilose Orafti) | 2.36 | g |
| Inulin (Raftiline Orafti) | 2.81 | g |
| Resistant starch (Novelose 330) | 2.0 | g |
| TOS (Vivinal, Borculo) | 0.25 | g |

| | |
|---|---|
| Maltodextrin | added to a final |
| | volume of 25 gram |

The above composition is intended as a daily dosage, which should be divided in two sachets and administered at two time points during the day. The contents of a sachet should be mixed with a cold liquid or other high water content composition e.g. water, milk, yogurt, etc. No hot liquids should be used.

### Example 7

### Shelf Life

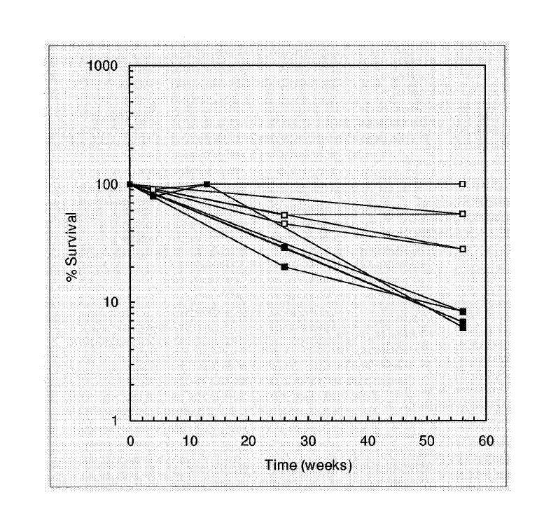

Freeze-dried Lactobacillus rhamnosus LMG P-22799 and Lactobacillus GG (ATCC 53103) were added to a powdered infant milk product (Nutrilon Pepti) at a start concentration of about 2×10⁸ cfu/g. The powder was added to cans, which were flushed with nitrogen and closed. The cans were stored at room temperature. At several time points a can was opened and samples were taken in which the concentration of surviving Lactobacilli was determined. For each time point a new can was opened. The product powder containing Lactobacillus was diluted into sterile PBS and plated in several dilutions on MRS-agar and incubated at 37° C. for 2 days. The percentage survival was determined by setting the concentration at t=0 at 100%. In FIG. 1 it is shown that strain Lactobacillus LMG P-22799 has a better survival than Lactobacillus GG.

### Example 8

### Antibiotic Sensitivity

### 8.1 Method of Determination of Antibiotic Resistance

The Minimal inhibitory concentration (MIC) was determined by serial dilution of the tested antibiotics in MRS broth (Oxoid) and inoculate the broth with the probiotic strain to be tested. The lowest concentration of the antibiotic resulting in no growth after 24 hours of incubation at 37° C. was set as the MIC.

The tested antibiotic were:

ampicillin (Boehringer Mannheim, 835242), bacitracin (Fluka, 11702), chloramphenicol Boehringer Mannheim, 634433), colistin sulphate (Fluka, 27655), erythromycin (Sigma, E5389), kanamycin (Sigma, K4000), lincomycin (Sigma, L2774), neomycin (Sigma, N1142), nystatin (Sigma, N4014), penicillin (Sigma, P3032), polymixin B sulphate (Merck, 1.06994.0005), streptomycin (Sigma, S2522), tetracyclin (Sigma, T8032), vancomycin (Vancomycin CP, Faulding Pharmaceuticals).

The following stock solutions were prepared and stored at −20° C.:

| | | |
|---|---|---|
| ampicillin | 0.04 | mg/ml demi water |
| bacitracin | 4 | mg/ml 0.01 N HCl |
| cloramphenicol | 0.04 | mg/ml EtOH abs. |
| colistin sulphate | 4 | mg/ml demi water |
| erythromycin | 0.4 | mg/ml demi water |
| kanamycin | 0.4 | mg/ml demi water |
| lincomycin | 0.04 | mg/ml demi water |
| neomycin | 0.4 | mg/ml 0.9% NaCl |
| nystatin | 4 | mg/ml demi water |
| penicillin | 0.04 | mg/ml demi water |
| polymixin B sulphate | 4 | mg/ml demi water |
| streptomycin | 0.04 | mg/ml demi water |
| tetracyclin | 0.04 | mg/ml demi water |
| vancomycin | 4 | mg/ml demi water |

### Sample Preparation:

Strains of Lactobacilli were cultured for 24 hours in MRS-broth at 37° C. and under aerobic conditions. A dilution series of the 24 hours-culture in MRS-broth was prepared (dilution factor 10, from 10⁻¹ to 1⁻¹⁰). The dilution series were cultured overnight (i.e. 16 h) at 37° C. under aerobic conditions. The highest dilution in which still growth was observed was selected to inoculate a 96-wells plate. The culture was centrifuge for 10 min at 3800 rpm and 4° C. and resuspended in the same volume of PBS (4° C.). The Lactobacilli were counted under a microscope and the solution was diluted in cold PBS to obtain a concentration of 1*10⁸ cells/ml.

### Assay:

Wells of a 96-wells plate were filled with MRS broth and with antibiotics in varying dilution to a total volume of 95 μl. As a control, no antibiotics were added. The following final dilutions of antibiotics were tested:

bacitracin, colistin, nystatin, polymixin B, vancomycin: 1000, 500, 250, 125, 62.5 ug/ml
erythromycin, kanamycin, neomycm: 100, 50, 25, 12.5, 6.25 ug/ml
ampicillin, chloramphenicol, lincomycin, penicillin, streptomycin, tetracyclin: 10, 5, 2.5, 1.25, 0.625 ug/ml

The plates were incubated for 24 hours at 37° C. in a SpectaMAX plate reader (shaking every 10 minutes for 20 seconds) and the optical density was determined at 600 nm.

### 8.2 Results—MIC

As shown in Table 5, L. rhamnosus LMG P-22799 is in general more sensitive to antibiotics than strain Lactobacillus rhamnosus GG. In Table 5 only the antibiotics to which strain LMG P-22799 was more sensitive are shown. For all other antibiotics tested the two strains were equally sensitive and/or resistant (data not shown).

TABLE 5
Antibiotic sensitivity (expressed as MIC) of
L. rhamnosus LMG P-22799 and L. rhamnosus GG (ATCC 53103).

| | L. rhamnosus | L. rhamnosus GG |
|---|---|---|
| Antibiotics | LMG P-22799 | (ATTC 53103) |
| Ampicillin (μg/ml) | 2.5 | >10 |
| Tetracyclin | 2.5 | 10 |
| Streptomycin | 5 | >10 |
| Lincomycin | 5 | 10 |
| Erythromycin | 50 | >100 |
| Chloramphenicol | 5 | >10 |

8.3 Broth Micro Dilution Test for 131 Different Lactobacillus rhamnosus Strains

The inocula of 131 strains of Lactobacillus rhamnosus species are prepared by suspending several freshly cultivated single colonies in a tube with 5 ml of saline up to an optical density of McFarland standard No. 0.5. The corresponding colonies are picked up from MRS (de Man, Rogosa, Sharpe) agar plates on which the strains grew for 48 h at 37° C. and at 5% CO₂ atmosphere. Subsequently, this suspension is diluted 1:10 by transferring 4 ml of the McFarland No. 0.5 suspension into a suitable inoculum container with 36 ml of saline and subsequent careful mixing. The MIC microtiter test plates (95 wels with different concentrations of the test antibiotics and one wel for the growth control without any antibiotic) were prepared before. Nutrient medium: LSM (lactobacilli susceptibility test medium) broth consisting of 90% Iso-sensitest broth plus 10% MRS broth (pH 6.7). The inoculations of the pre-made MIC test plates are performed by a multipoint inoculator (final inoculum of these LAB in the microtiter plate: about 10⁵ bacteria ml⁻¹). The plates are subsequently incubated in ambient air or in a 5% CO₂ atmosphere at 37° C. for 24 (−48) h. Then the MICs are read as the lowest concentration in the increasing concentration row of the corresponding antibiotic in which no growth of the test organism was observed. The control attempt (without antibiotic) of the corresponding strain tested has to show sufficient growth. Table 6 shows the results as the number of stains within a dilution range 1, 2, 4, 8, 16, 32, 64 that minimally inhibits the growth of the test organisms.

TABLE 6

| | Number of strains with a MIC (μg/ml) | | | | | | |
|---|---|---|---|---|---|---|---|
| Antibiotic | 0.125 | 0.25 | 0.5 | 1.0 | 2.0 | 4.0 | 8.0 |
| Penicillin | 9 | 76 | 28 | 11 | 7 | | |
| AMP | | 3 | 52 | 62 | 12 | 2 | |
| ASU | | 3 | 51 | 64 | 11 | 2 | |
| GEN | | | | 95 | 33 | 2 | 1 |
| STR | | | | | 37 | 52 | 21 |
| Q/D | 29 | 85 | 13 | 3 | | | |
| OTE | 2 | 45 | 78 | 2 | | | 4 |
| CMP | | | 1 | 10 | 88 | 31 | 1 |

In a large study comprising 131 different Lb. rhamosus strains the sensitivity for antibiotics was tested. In table 6 the number of strains falling within a certain dilution factor are shown. Clearly there are large differences in antibiotic sensitivity within the Lb. rhamnosus group. The more sensitive to these antibiotics, the better and together with the other parameters, such as the inhibition of adhesion of pathogens to intestinal epithelial cells is used to select the most appropriate Lb. rhamnosus. For ampiciline (AMP) our preferred Lb. rhamnosus belongs to the group with a MIC ≦0.5 while Lb. rhamnosus GG belongs to the larger group with a MIC ≧1.0, clearly indicating the distinguishing value of this parameter. The AMP sensitivity ratio LGG/LMG P-22799=2-4.

1-16. (canceled)

17. An enteral composition for the treatment and/or prevention of sepsis, bacteraemia and/or endotoxaemia in humans having surgery, comprising a strain of Lactobacillus rhamnosus, wherein less than 5% of the lactate produced by the strain is D-lactate, determined enzymatically using an L-lactate acid detection kit with D- and L-lactate-dehydrogenase, and wherein the strain is at least 2 times as sensitive to ampicillin, chloramphenicol, or both as is a strain of L. rhamnosus GG.

18. The composition according to claim 17 further comprising a strain of Bifidobacterium selected from the group consisting of B. breve, B. lactis, B. longum, B. animalis, B. adolescentis, B. infantis, and B. bifidum.

19. The composition according to claim 17 further comprising a non-digestible carbohydrate selected from the group consisting of galacto-oligosaccharides, trans-galacto-oligosaccharides, fructo-oligosaccharides, inulin, xylo-oligosaccharides, pectin oligosaccharides, partially hydrolysed galactomannan and indigestible polydextrose.

20. The composition according to claim 19, wherein the non-digestible carbohydrate is trans-galacto-oligosaccharides.

21. The composition according to claim 20 comprising 0.2 to 25 g trans-galacto-oligosaccharides per 10⁸ to 10¹¹ cfu L. rhamnosus.

22. The composition according to claim 17 further comprising a yeast of the genus Saccharomyces.

23. The composition according to claim 17 further comprising a digestible, soluble carbohydrate.

24. The composition according to claim 23 comprising 5 to 230 g digestible, soluble carbohydrates per dose.

25. The composition according to claim 17 comprising 1×10⁶ to 1×10¹² colony forming units of strain Lactobacillus rhamnosus per daily dose

26. The composition according to claim 25 comprising 1×10⁸ to 5×10¹⁰ colony forming units of strain Lactobacillus rhamnosus per daily dose.

27. The composition according to claim 17, wherein the strain of L. rhamnosus strain has accession number LMG P-22799 or a derivative thereof.

28. A method for the treatment and/or prevention of sepsis, bacteraemia and/or endotoxaemia in humans having surgery, the method comprising administering to said patient in need thereof a dose of a composition comprising a strain of Lactobacillus rhamnosus, wherein less than 5% of the lactate produced by the strain is D-lactate, determined enzymatically using an L-lactate acid detection kit with D- and L-lactate-dehydrogenase, and wherein the strain is at least 2 times as sensitive to ampicillin, chloramphenicol, or both as is a strain of L. rhamnosus GG.

29. The method according to claim 28, wherein said composition is administered before surgery.

30. The method according to claim 28, wherein said composition is administered after surgery.

31. The method according to claim 28, wherein the composition is administered during surgery.

32. The method according to claim 28, wherein the surgery is abdominal surgery or cardio-surgery.

33. The method according to claim 28, wherein the composition is administered at least once at least 12 hours before and/or surgery.

34. The method according to claim 28, wherein the composition is administered at least once a day at least four days before and/or after surgery.

35. The method according to claim 28, wherein the composition is administered at least twice a day for at least two days before and/of after surgery.

36. A pharmaceutical composition comprising the strain of L. rhamnosus strain has accession number LMG P-22799 or a derivative thereof.

37. The pharmaceutical composition according to claim 36, wherein said composition is an enteral composition.

38. A method for the treatment and/or prevention of sepsis, bacteraemia and/or endotoxaemia in a human, the method comprising administering to said patient in need thereof a dose of a composition comprising a strain of L. rhamnosus strain has accession number LMG P-22799 or a derivative thereof.

39. The method according to claim 38, wherein the bacteraemia and/or endotoxaemia is inflammatory bowel disease, pouchitis, pancreatitis, liver cirrhosis or liver failure, necrotizing enterocolitis, burns, allergy, or an intestinal infection caused by entero-invasive pathogens a permeable intestine due to anticancer therapy.
